(19) United States
(12) Patent Application Publication
(10) Pub. No.: US 2025/0152560 A1
(43) Pub. Date: May 15, 2025

(54) HDAC INHIBITOR OKI-179 IN COMBINATION WITH BINIMETINIB FOR THE TREATMENT OF CANCER

(71) Applicants: OnKure, Inc., Boulder, CO (US); Array BioPharma Inc., Boulder, CO (US)

(72) Inventors: Anthony D. Piscopio, Boulder, CO (US); Patrice Anne Lee, Boulder, CO (US); James Winkler, Boulder, CO (US); Richard D. Woessner, Boulder, CO (US)

(73) Assignees: OnKure, Inc., Boulder, CO (US); Array BioPharma Inc., Boulder, CO (US)

(21) Appl. No.: 18/838,130

(22) PCT Filed: Feb. 13, 2023
(86) PCT No.: PCT/US2023/012909

§ 371 Date: Aug. 13, 2024

Related U.S. Application Data
(60) Provisional application No. 63/408,261, filed on Sep. 20, 2022.
(60) Provisional application No. 63/310,868, filed on Feb. 16, 2022.

(51) Int. Cl. A61K 31/429; A61K 31/4184; A61K 31/506; A61P 35/00

(52) U.S. Cl. CPC A61K 31/429; A61K 31/4184; A61K 31/506; A61P 35/00

(57) ABSTRACT

A method of treating cancer in a subject in need thereof, wherein the method comprises administration of therapeutically effective amounts of OKI-179 and binimetinib or a pharmaceutically acceptable salt thereof, or administration of therapeutically effective amounts of OKI-179, binimetinib or a pharmaceutically acceptable salt thereof, and encorafenib or a pharmaceutically acceptable salt thereof according to the dosing schedules described herein.

### CROSS-REFERENCE TO RELATED APPLICATIONS

This application claims the priority benefit of U.S. Provisional Application No. 63/310,868, filed on Feb. 16, 2022 and U.S. Provisional Application No. 63/408,261 filed on Sep. 20, 2022, each of which is hereby incorporated by reference in its entirety.

### BACKGROUND OF THE INVENTION

It is well known that the RAS pathway is frequently activated in human cancers and thus may provide multiple opportunities for therapeutic interventions. Activated growth factor receptors signal through RAS, as well as other pathways, and RAS mutations account for half or more of colorectal cancers and lung cancers and significant fractions of other cancers (Sanchez-Vega et al., Cell. 173(2): 321-337.e10. doi: 10.1016/j.cell.2018.03.035. PMID: 29625050; PMCID: PMC6070353(2018). Activating RAS pathway mutations are found in greater than 30% of human tumors and are often associated with poor outcomes. While RAS-pathway targeted drugs have been approved, their activities as single-agents remain modest, supporting the need for rational targeted combinations to improve patient outcomes.

Multiple studies have proposed a chemical synthetic lethality between Class I histone deacetylase inhibitors (HDACi), and RAS-pathway inhibitors in RAS-pathway mutated models (Maertens et al., Cancer Discovery 9, 526-545 (2019); Yamada et al., Mol. Cancer Ther. 17, 17-25 (2018); Faiao-Flores et al., Melanoma Manag. 6(4): MMT29. doi: 10.2217/mmt-2019-0017 (2019); Ischenko et al., Oncotarget. 6(18), 15814-27 (2015); Wang et al., Cell 173, 1413-25 (2018); Chao et al., Clin Epigenetics 11(1): 85. doi: 10.1186/s13148-019-0681-6 (2019); Bahr et al., Oncotarget 7(43), 69804-69815 (2016)). This synthetic lethality occurs due to a drug combination effect and results in the inhibition of double-stranded (ds) DNA repair and other survival pathways which drive apoptosis and tumor regressions. The use of HDACi in solid tumors and in combination therapies has been impeded by poor potency, lack of selectivity, safety issues and dosing inconvenience, thus highlighting the need for a better HDACi in the clinic.

OKI-179, a novel largazole derivative, is a potent, Class I-selective, oral HDACi that has completed Phase 1 clinical trials as a single agent in patients with solid tumors. The clinical profile of OKI-179 shows its potential to achieve exposure consistent with preclinical activity with strong pharmacodynamic activity at tolerated doses, supporting the development of OKI-179 for use in solid tumor combinations.

### SUMMARY OF THE INVENTION

An aspect of the present invention is a method of treating cancer by administering to a subject in need thereof therapeutically effective amounts of OKI-179 and the MEK inhibitor binimetinib (5-[(4-bromo-2-fluorophenyl) amino]-4-fluoro-N-(2-hydroxyethoxy)-1-methyl-1H-benzimidazole-6-carboxamide) (MEKTOVI®) (“bini”) or a pharmaceutically acceptable salt thereof according to a dosing schedule comprising at least one 7-day dosing cycle, wherein OKI-179 is administered once per day (QD) on days 1-4 of said 7-day dosing cycle and binimetinib is administered twice per day (BID) on days 1-7 of said 7-day dosing cycle.

Another aspect of the present invention is a method of treating cancer by administering to a subject in need thereof therapeutically effective amounts of OKI-179 and binimetinib or a pharmaceutically acceptable salt thereof according to a dosing schedule comprising at least one 7-day dosing cycle, wherein OKI-179 is administered once per day (QD) on days 1-5 of said 7-day dosing cycle and binimetinib is administered twice per day (BID) on days 1-7 of said 7-day dosing cycle.

Another aspect of the present invention is a method of treating cancer by administering to a subject in need thereof therapeutically effective amounts of OKI-179, binimetinib or a pharmaceutically acceptable salt thereof, and the BRAF inhibitor encorafenib (methyl N-{(2S)-1-[(4-{3-[5-chloro-2-fluoro-3-(methanesulfonamido)phenyl]-1-(propan-2-yl)-1H-pyrazol-4-yl}pyrimidin-2-yl)amino]propan-2-yl}carbamate) (BRAFTOVI®) (“enco”) or a pharmaceutically acceptable salt thereof according to a dosing schedule comprising at least one 7-day dosing cycle, wherein OKI-179 is administered once per day (QD) on days 1-4 of said 7-day dosing cycle, binimetinib is administered twice per day (BID) on days 1-7 of said 7-day dosing cycle and encorafenib is administered once per day (QD) on days 1-7 of said 7-day dosing cycle.

Another aspect of the present invention is a method of treating cancer by administering to a subject in need thereof therapeutically effective amounts of OKI-179, binimetinib or a pharmaceutically acceptable salt thereof, and encorafenib or a pharmaceutically acceptable salt thereof according to a dosing schedule comprising at least one 7-day dosing cycle, wherein OKI-179 is administered once per day (QD) on days 1-5 of said 7-day dosing cycle, binimetinib is administered twice per day (BID) on days 1-7 of said 7-day dosing cycle and encorafenib is administered once per day (QD) on days 1-7 of said 7-day dosing cycle.

In an embodiment, the cancer is a NRAS-mutated cancer or a BRAF-mutated cancer. In an exemplary embodiment, the cancer is a NRAS-mutated melanoma, a BRAF-mutated melanoma or a BRAF-mutated colorectal cancer.

In an embodiment, the subject is a human.

In an embodiment, OKI-179 and binimetinib are administered within 30 minutes of each other.

In an embodiment, OKI-179, binimetinib and encorafenib are administered within 30 minutes of each other.

In an embodiment, OKI-179, binimetinib and encorafenib are administered orally.

Another aspect of the invention is a pharmaceutical combination comprising OKI-179 and binimetinib or a pharmaceutically acceptable salt thereof.

Another aspect of the invention is a pharmaceutical combination comprising OKI-179, binimetinib or a pharmaceutically acceptable salt thereof, and encorafenib or a pharmaceutically acceptable salt thereof.

### BRIEF DESCRIPTION OF THE DRAWINGS

FIG. 1 illustrates that the combination of OKI-005 and binimetinib was observed to synergistically inhibit NRASₘᵤₜSK-Mel-2 cell growth.

FIGS. 2A, 2B and 2C illustrate the results observed in Protocol 1 as described herein.

FIGS. 3A, 3B and 3C illustrate the results observed in Protocol 3 as described herein.

FIGS. 4A, 4B and 4C illustrate the results observed in Protocol 4 as described herein.

FIGS. 5A, 5B and 5C illustrate the results observed in Protocol 5 as described herein.

FIG. 6A illustrates that the combination of OKI-005 and binimetinib was observed to synergistically inhibit NRASₘᵤₜSK-Mel-2 melanoma cell growth.

FIG. 6B illustrates this synergy by a comparison of the CTG cell viability signal from Day 0 to Day 3. FIG. 6C illustrates this synergy by measuring cell death via increasing PARP cleavage over time.

FIG. 7 illustrates the synergistic effects in MGMT-positive patients versus MGMT-negative patients when dosed with the combination of OKI-179 and binimetinib compared to OKI-179 or binimetinib alone.

### DETAILED DESCRIPTION OF THE INVENTION

### Definitions

As defined herein, “OKI-179” refers to the compound of the following structure that exists as a benzenesulfonate salt:

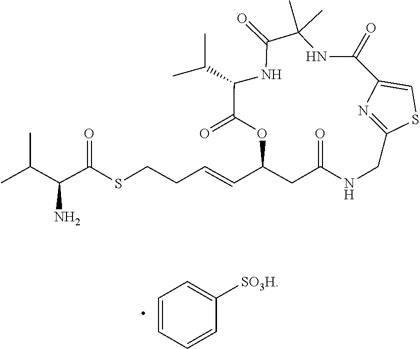 [SMILES: CC(C)[C@H](N)C(=O)SCC/C=C/[C@@H]1CC(=O)NCc2nc(cs2)C(=O)NC(C)(C)C(=O)N[C@@H](C(C)C)C(=O)O1 ; O=S(=O)(O)c1ccccc1]

As defined herein, “OKI-005” refers to the compound of the following structure:

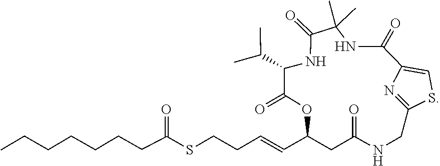 [SMILES: CCCCCCCC(=O)SCC/C=C/[C@@H]1CC(=O)NCc2nc(cs2)C(=O)NC(C)(C)C(=O)N[C@@H](C(C)C)C(=O)O1]

As defined herein, “bini” refers to the MEK inhibitor binimetinib.

As defined herein, “enco” refers to the BRAF-inhibitor encorafenib.

As defined herein, “bini/enco” refers to the combination of bini and enco.

As defined herein, “CDX” refers to cell-line derived xenograft.

As defined herein, “PDX” refers to patient-derived xenograft.

As defined herein, “BWL” refers to body weight loss.

As defined herein, “TGI” refers to tumor growth inhibition.

As defined herein, “QD” refers to once per day dosing.

As defined herein, “BID” refers to twice per day dosing.

As defined herein, “PO” refers to oral (“by mouth”) administration.

As defined herein, “mpk” refers to milligrams per kilogram (mg/kg).

As defined herein, “R” refers to regression and reflects an observed decrease in tumor size.

As defined herein, “QW” refers to once per week dosing.

As defined herein, “LOF” refers to loss of function.

As defined herein, the term “subject” includes, but is not limited to, humans (e.g., a male or female of any age group, e.g., a pediatric subject (e.g., infant, child, adolescent) or adult subject (e.g., young adult, middle-aged adult or senior adult)) and/or other primates (e.g., monkeys); non-human mammals, such as cows, pigs, horses, sheep, mice, goats, cats, and/or dogs; and/or birds, such as chickens, ducks and/or geese.

As defined herein, “cancer” refers to a physiological condition in mammals that is typically characterized by unregulated cell growth. Examples of cancer include, but are not limited to, carcinoma, lymphoma, leukemia, blastoma, and sarcoma.

In one embodiment, the cancer is a BRAF-associated cancer. The term “BRAF-associated cancer” as used herein refers to cancers associated with or having a BRAF V600 mutation, e.g., a BRAF V600E, V600K, V600R or V600S mutation. In one embodiment, the BRAF-associated cancer is a cancer having a BRAF V600E mutation. Non-limiting examples of BRAF-associated cancers are described herein.

In one embodiment, the cancer is an NRAS-associated cancer. The term “NRAS-associated cancer” as used herein refers to cancers associated with or having an NRAS Q61 mutation, e.g., an NRAS Q61H, Q61K, Q61L or Q61R mutation. In one embodiment, the NRAS-associated cancer is a cancer having an NRAS Q61K mutation. In one embodiment, the NRAS-associated cancer is a cancer having an NRAS Q61L mutation. In one embodiment, the NRAS-associated cancer is a cancer having an NRAS Q61R mutation. Non-limiting examples of NRAS-associated cancers are described herein.

More particular examples of such cancers include squamous cell carcinoma, small cell lung cancer, non-small cell lung cancer (NSCLC) (including, but not limited to, metastatic non-small cell lung cancer, BRAF-mutated NSCLC (e.g., BRAF V600E mutated NSCLC), glioma, Hodgkin's lymphoma, non-Hodgkin's lymphoma, acute myeloid leukemia (AML), multiple myeloma, gastrointestinal cancer, renal cell carcinoma, renal cancer (e.g., advanced renal cell carcinoma), ovarian cancer, liver cancer, lymphoblastic leukemia, lymphocytic leukemia, colorectal cancer (including, but not limited to, metastatic colorectal cancer (e.g., microsatellite stable metastatic colorectal cancer), BRAF V600 mutant colorectal cancer (e.g., BRAF V600E or BRAF V600K mutant colorectal cancer), endometrial cancer, kidney cancer, prostate cancer, thyroid cancer, melanoma (including, but not limited to, unresectable or metastatic melanoma, uveal melanoma, BRAF V600 mutant melanoma, such as BRAF V600E mutant melanoma), chondrosarcoma, neuroblastoma, pancreatic cancer, glioblastoma multiforme, cervical cancer, brain cancer, stomach cancer, urothelial carcinoma (including local advanced or metastatic urothelial carcinoma), bladder cancer, hepatoma, breast cancer and head and neck cancer. In one embodiment, the cancer is colorectal cancer. In one embodiment, the cancer is metastatic colorectal cancer. In one embodiment, the cancer is melanoma. In another embodiment, the cancer is pancreatic cancer. In another embodiment, the cancer is NSCLC. In another embodiment, the cancer is NRAS-mutated melanoma or BRAF-mutated melanoma.

As defined herein, the phrase “combination therapy” refers to refers to a dosing regimen of two or more different therapeutically active agents during a period of time, wherein the therapeutically active agents are administered together or separately in a manner. In one embodiment the combination therapy is a non-fixed combination.

The term “non-fixed combination” means that the two or more different therapeutic agents are formulated as separate compositions or dosages such that they may be administered separately to a subject in need thereof either simultaneously or sequentially with variable intervening time limits.

As defined herein, the phrase “effective dosage” or “effective amount” or “therapeutically effective amount” of a compound or a pharmaceutical composition is an amount sufficient to result in any one or more beneficial or desired results in a subject.

As defined herein, the term “synergy” or “synergistic” refers to the phenomenon where the combination of two therapeutic agents of a combination therapy is greater in terms of measured results than the sum of the effect of each agent when administered alone.

As defined herein, the term “in vivo” refers to an event that takes place in a subject's body.

As defined herein, the term “in vitro” refers to an event that takes places outside of a subject's body.

As defined herein, a “pharmaceutically acceptable form” of a compound includes, but is not limited to, pharmaceutically acceptable salts, hydrates, solvates, isomers, prodrugs, and isotopically labeled derivatives of a compound.

As defined herein, the term “pharmaceutically acceptable salt” refers to salts that do not adversely impact the biological activity and properties of the compound and are suitable for use in contact with the tissues of subjects without undue toxicity, irritation and/or allergic response and the like. Pharmaceutically acceptable salts include those derived from suitable inorganic acids, organic acids and bases, and include hydrochloric acid, hydrobromic acid, phosphoric acid, sulfuric acid, nitric acid, acetic acid, oxalic acid, maleic acid, tartaric acid, citric acid, succinic acid, malonic acid, ascorbic acid, methanesulfonic acid, ethanesulfonic acid, benzene sulfonic acid, p-toluenesulfonic acid, benzoic acid, naphthalene sulfonic acid, lactic acid, succinic acid, oxalic acid, stearic acid, and the like. In some instances, pharmaceutically acceptable salts are obtained by reacting a compound having acidic group described herein with a base to form a salt such as an ammonium salt, an alkali metal salt (e.g., a sodium or a potassium salt), an alkaline earth metal salt (e.g., a calcium or a magnesium salt), a salt formed from an organic base, and an amino acid salt. Pharmaceutically acceptable salts derived from appropriate bases include alkali metals, alkaline earth metals, and ammonium and quaternary ammonium compounds. Specific metals include, but are not limited to, sodium, lithium, potassium, calcium, magnesium, iron, zinc, copper, manganese, aluminum, and the like. Organic bases from which salts may be prepared include, for example, primary, secondary, and tertiary amines.

As defined herein, the term “prodrug” refers to a compound that is transformed in vivo to yield a disclosed compound or a pharmaceutically acceptable form of the compound. A prodrug may be inactive when administered to a subject, but is converted in vivo to an active compound. In various instances, a prodrug has improved physicochemical properties (such as bioavailability) and/or delivery properties over the parent compound. Prodrugs are typically designed to enhance pharmaceutically and/or pharmacokinetically based properties associated with the parent compound. The prodrug compound often offers advantages of solubility, tissue compatibility or delayed release in subject. Prodrugs include compounds wherein a hydroxy, amino or mercapto group is bonded to any group that, when the prodrug is administered to a subject, cleaves to form a free hydroxy, free amino or free mercapto group, respectively. See, e.g., Bundgaard, H., Design of Prodrugs (1985) (Elsevier, Amsterdam).

As defined herein, the phrase “treat” or “treating” a cancer means to administer a compound of the present invention to a subject having cancer or having been diagnosed with cancer, to achieve at least one positive therapeutic effect, such as, for example, reduced number of cancer cells, reduced tumor size, reduced rate of cancer cell infiltration into peripheral organs, or reduced rate of tumor metastases or tumor growth, reversing, alleviating, or inhibiting the progress of, the disorder or condition to which such term applies, or one or more symptoms of such disorder or condition. The term “treatment”, as used herein, unless otherwise indicated, refers to the act of treating as “treating” is defined immediately above. The term “treating” also includes adjuvant and neo-adjuvant treatment of a subject.

For the purposes of this invention, beneficial or desired clinical results include, but are not limited to, one or more of the following: reducing the proliferation of (or destroying) neoplastic or cancerous cell; inhibiting metastasis or neoplastic cells; shrinking or decreasing the size of a tumor; an increase in the period of remission for a subject (e.g., as compared to the one or more metric(s) in a subject having a similar cancer receiving no treatment or a different treatment, or as compared to the one or more metric(s) in the same subject prior to treatment); decreasing symptoms resulting from the cancer; increasing the quality of life of those suffering from the cancer; decreasing the dose of other medications required to treat the cancer; delaying the progression of the cancer; curing the cancer; overcoming one or more resistance mechanisms of the cancer; and/or prolonging survival of patients the cancer. Positive therapeutic effects in cancer can be measured in several ways (see, e.g., W. A. Weber, J. Nucl. Med. 50 Suppl. 1:1 S-10S (2009)). For example, with respect to tumor growth inhibition (T/C), according to the National Cancer Institute (NCI) standards, a T/C less than or equal to 42% is the minimum level of anti-tumor activity. A T/C<10% is considered a high anti-tumor activity level, with T/C (%)=median tumor volume of the treated/median tumor volume of the control×100.

In one embodiment, the treatment achieved by administration of a compound of the invention is defined by reference to any of the following: partial response (PR), complete response (CR), overall response (OR), progression free survival (PFS), disease free survival (DFS) and overall survival (OS). PFS, also referred to as “Time to Tumor Progression” indicates the length of time during and after treatment that the cancer does not grow and includes the amount of time patients have experienced a CR or PR, as well as the amount of time patients have experienced stable disease (SD). DFS refers to the length of time during and after treatment that the patient remains free of disease. OS refers to a prolongation in life expectancy as compared to naive or untreated subjects or patients. In one embodiment, response to treatment with a compound of the invention is any of PR, CR, OR, PFS, DFS, or OS that is assessed using Response Evaluation Criteria in Solid Tumors (RECIST) 1.1 response criteria.

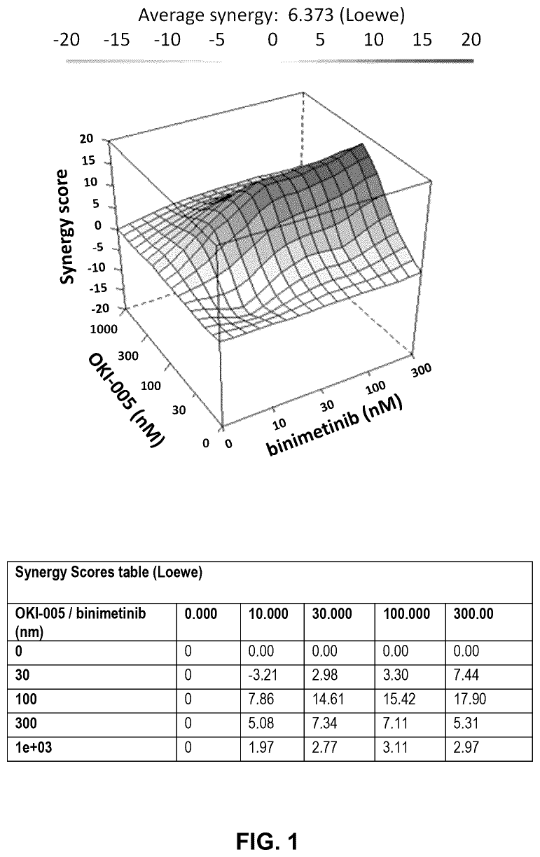

It was unexpectedly observed that the combination of OKI-179 with binimetinib (bini) and the combination of OKI-179 with bini and the encorafenib (enco) were each synergistic as assessed in both CDX and PDX models of NRAS-mutated melanoma and BRAF-mutated colorectal cancer (CRC). Combining OKI-179 with bini in vitro in the presence of SKMEL-2 NRASMT melanoma cells induced a significant increase in cell death compared to each of OKI-179 and bini as single agents at clinically achievable drug concentrations. FIG. 1 demonstrates that compound OKI-005 with an IC50 of 122 nm is a potent inhibitor of NRAS^{mut}SK-Mel-2 cell growth. However, the combination of OKI-005 and binimetinib was observed to synergistically inhibit NRASₘᵤₜSK-Mel-2 cell growth at clinically achievable concentrations as shown in the synergy scores table of FIG. 1.

The activity of OKI-179+bini and OKI-179+bini+enco was further investigated in vivo in established PDX models of NRAS-mutant melanoma, MEL278 and MM415, and in a CDX model of NRAS-mutant melanoma, SKMEL2. As single agents, both bini (at 3.5 mg/kg PO bid, 21 d) and OKI-179 (at 80 mg/kg PO; 3 weeks, dosed on days 1-5 of each week) doses that mimic the clinical exposure of these molecules, exhibited modest tumor growth inhibition (TGI). However, when bini and OKI-179 were combined in a dosing schedule where OKI-179 was administered either once per day (QD) on days 1-4 or once per day (QD) on days 1-5 of a 7-day dosing cycle and binimetinib was administered twice per day (BID) on days 1-7 of the 7-day dosing cycle, the observed result was a significantly greater TGI, including regression in 75% to 100% of the tumors after 14 or 15 days of treatment, as compared to 0% to 25% for bini alone. A similar outcome was also observed in HT29 tumors (BRAF^{mt} CRC) using the same dosing schedules where the combination of OKI-179 and enco+bini showed regression in 71% of tumors, compared to 0% for OKI-179 or 13% for enco+bini alone. No additivity and no regressions were observed in the CRC563 tumor model (BRAF^{mt}). The data also shows the potential for OKI-179 to synergize with binimetinib or with binimetinib+encorafenib across multiple BRAF-mutant and NRAS-mutant cancers. Table 1 below summarizes these observed results.

TABLE 1

| | RAS | | % Tumors with |
|---|---|---|---|
| | Pathway | | Regression |
| Model | Mutation | Treatment | at day 14/15 |
| HT29 | BRAF | bini + enco | 13% |
| (CRC) | | OKI-179 | 0% |
| Protocol 1 | | OKI-179 + | 71% |
| | | bini/enco | |
| MEL278 | NRAS | bini | 0% |
| (Melanoma) | | OKI-179 | 0% |
| Protocol 4 | | OKI-179 + bini | 100% |
| MM415 | NRAS | bini | 25% |
| (Melanoma) | | OKI-179 | 0% |
| Protocol 3 | | OKI-179 + bini | 75% |
| SKMEL2 | NRAS | bini | 0% |
| (Melanoma) | | OKI-179 | 0% |
| Protocol 5 | | OKI-179 + bini | 86% |
| CRC563 | BRAF | bini | 0% |
| (CRC) | | OKI-179 | 0% |
| Protocol 2 | | OKI-179 + | 0% |
| | | bini/enco | |

In an exemplary embodiment, the pharmaceutical compositions used as described herein further comprise one or more pharmaceutically acceptable excipients or diluents.

Because the combination comprising OKI-179 and binimetinib, and the combination comprising OKI-179 and binimetinib and encorafenib were observed to be synergistic (e.g., had a synergistic effect in treating or managing a cancer), the amount or dosage of OKI-179, binimetinib, or both as employed herein may be lower (e.g., by at least 10% or at least 20% or at least 30% or at least 40% or at least 50%) than the amount or dosage of OKI-179 and binimetinib used individually as employed herein, e.g., as in a monotherapy. Similarly, in various embodiments, the amount or dosage of one or more of OKI-179, binimetinib, encorafenib as employed herein when used in combination may be lower (e.g., by at least 10% or at least 20% or at least 30% or at least 40% or at least 50%) than the amount or dosage of OKI-179, binimetinib and encorafenib used individually as employed herein, e.g., as in a monotherapy.

Accordingly, in one embodiment provided herein is a method for treating cancer by administering to a subject in need thereof therapeutically effective amounts of OKI-179 and binimetinib or a pharmaceutically acceptable salt thereof.

In one embodiment, provided herein is a method of treating cancer by administering to a subject in need thereof therapeutically effective amounts of OKI-179 and binimetinib or a pharmaceutically acceptable salt thereof according to a dosing schedule comprising at least one 7-day dosing cycle, wherein OKI-179 is administered once per day (QD) on days 1-4 of said 7-day dosing cycle and binimetinib or a pharmaceutically acceptable salt thereof is administered twice per day (BID) on days 1-7 of said 7-day dosing cycle.

In one embodiment, provided herein a method of treating cancer by administering to a subject in need thereof therapeutically effective amounts of OKI-179 and binimetinib or a pharmaceutically acceptable salt thereof according to a dosing schedule comprising at least one 7-day dosing cycle, wherein OKI-179 is administered once per day (QD) on days 1-5 of said 7-day dosing cycle and binimetinib or a pharmaceutically acceptable salt thereof is administered twice per day (BID) on days 1-7 of said 7-day dosing cycle.

In one embodiment of any of the methods of treatment described herein, OKI-179 and binimetinib or a pharmaceutically acceptable salt thereof are formulated as separate dosages. In one embodiment, a dosage of OKI-179 is administered to the subject within 30 minutes of administration of a dosage of binimetinib or a pharmaceutically acceptable salt thereof. In another embodiment, a dosage of OKI-179 is administered to the subject within 60 minutes of administration of a dosage of binimetinib or a pharmaceutically acceptable salt thereof.

In one embodiment provided herein is a method for treating cancer by administering to a subject in need thereof therapeutically effective amounts of OKI-179, binimetinib or a pharmaceutically acceptable salt thereof, and encorafenib or a pharmaceutically acceptable salt thereof.

In one embodiment, provided herein is a method of treating cancer by administering to a subject in need thereof therapeutically effective amounts of OKI-179, binimetinib or a pharmaceutically acceptable salt thereof, and encorafenib or a pharmaceutically acceptable salt thereof, according to a dosing schedule comprising at least one 7-day dosing cycle, wherein OKI-179 is administered once per day (QD) on days 1-4 of said 7-day dosing cycle, binimetinib or a pharmaceutically acceptable salt thereof is administered twice per day (BID) on days 1-7 of said 7-day dosing cycle, and encorafenib or a pharmaceutically acceptable salt thereof is administered once per day (QD) on days 1-7 of said 7-day dosing cycle.

In one embodiment, provided herein is a method of treating cancer by administering to a subject in need thereof therapeutically effective amounts of OKI-179, binimetinib or a pharmaceutically acceptable salt thereof, and encorafenib or a pharmaceutically acceptable salt thereof, according to a dosing schedule comprising at least one 7-day dosing cycle, wherein OKI-179 is administered once per day (QD) on days 1-5 of said 7-day dosing cycle, binimetinib or a pharmaceutically acceptable salt thereof is administered twice per day (BID) on days 1-7 of said 7-day dosing cycle, and encorafenib or a pharmaceutically acceptable salt thereof is administered once per day (QD) on days 1-7 of said 7-day dosing cycle.

In one embodiment of any of the methods of treatment described herein, OKI-179, binimetinib or a pharmaceutically acceptable salt thereof, and encorafenib or a pharmaceutically acceptable salt thereof are formulated as separate dosages. In one embodiment, a dosage of OKI-179 is administered to the subject within 30 minutes of administration of a dosage of binimetinib or a pharmaceutically acceptable salt thereof and a dosage of encorafenib or a pharmaceutically acceptable salt thereof. In another embodiment, a dosage of OKI-179 is administered to the subject within 60 minutes of administration of a dosage of binimetinib or a pharmaceutically acceptable salt thereof and a dosage of encorafenib or a pharmaceutically acceptable salt thereof.

In one embodiment, provided herein is a pharmaceutical combination comprising therapeutically effective amounts of OKI-179 and binimetinib or a pharmaceutically acceptable salt thereof. In one embodiment of the pharmaceutical combination, OKI-179 and binimetinib or a pharmaceutically acceptable salt thereof are formulated as separate dosages.

In one embodiment, provided herein is a pharmaceutical combination comprising therapeutically effective amounts of OKI-179, binimetinib or a pharmaceutically acceptable salt thereof, and encorafenib or a pharmaceutically acceptable salt thereof. In one embodiment of the pharmaceutical combination, OKI-179, binimetinib or a pharmaceutically acceptable salt thereof, and encorafenib or a pharmaceutically acceptable salt thereof, are formulated as separate dosages.

### EXAMPLES

### Dosing Protocols

The combination of OKI-179 with binimetinib (bini) and the combination of OKI-179 with binimetinib and encorafenib (bini/enco) was investigated in immunocompromised mice using well-characterized human tumor xenograft CDX and PDX models. In particular, the combination of OKI-179 and bini using NRAS melanoma xenografts and the combination of OKI-179 and bini/enco using BRAF V600E melanoma or CRC xenografts were tested. For bini only studies, the dosage was 3.5 mpk bini BID. For bini/enco studies, the dosage was 3.5 mpk bini BID and 20 mpk enco QD. Because of possible tolerance challenges associated with OKI-179, animals had their body weight checked before dosing whenever possible.

### Protocol 1

### HT-29 (BRAF V600E) Protocol

The OKI-179/bini/enco HT-29 (BRAF V600E) xenograft study was set up as follows:

### Study Design

vehicle control BID (days 1-21);
3.5 mpk bini BID (days 1-21)/20 mpk enco QD (days 1-21)
80 mpk OKI-179 QD1 (5 consecutive days on/2 consecutive days off for three weeks)
3.5 mpk bini BID (days 1-21)/20 mpk enco QD (days 1-21)/80 mpk OKI-179 QD1 (5 consecutive days on/2 consecutive days off for three weeks)
3.5 mpk bini BID (days 1-21)/20 mpk enco QD (days 1-21)/40 mpk OKI-179 QD1 (5 consecutive days on/2 consecutive days off for three weeks).

### Dosing Solutions

bini (100% active): Bini was added to 1% CMC/0.5% Tween 80 to make a concentration of 0.35 mg/mL. The suspension was sonicated for 30 minutes and stored at 4° C.
bini (100% active)/enco (100% active): 1% CMC/0.5% Tween 80 was added to bini to make a concentration of 0.7 mg/ml and the suspension was sonicated for 30 minutes. 1% CMC/0.5% Tween 80 was added to enco to make a concentration of 4 mg/mL and the suspension was sonicated for 30 minutes. The bini and enco suspensions were mixed 1:1 for a final dosing suspension of 0.35 mg/mL bini and 2 mg/mL enco and the combined suspension was stored at 4° C.
OKI-179 (100% active):
   Citrate buffer solution: Solution A comprising 0.1M citric acid and Solution B comprising 0.1M citric acid, tri-sodium were combined (100 parts Solution A: 20 parts Solution B) to provide citrate buffer pH3.
   80 mg/kg OKI-179: Dried OKI-179 was added to citrate buffer pH3 and stirred until a homogenous formulation of 8 mg/ml was obtained. The dose solution was stored at 4° C. and use within 10 days.

Table 2 below summarizes the above dosing solutions.

TABLE 2

| | | Dose | Dose | Conc. | | |
|---|---|---|---|---|---|---|
| API | % API | mg/kg | ml/kg | mg/ml | Vehicle | pH |
| bini | 100 | 3.5 | 10 | 0.35 | 1% CMC/0.5% | |
| | | | | | Tween 80 | |
| enco | 100 | 20 | 10 | 2 | 1% CMC/0.5% | |
| | | | | | Tween 80 | |
| OKI-179 | 100 | 80 | 10 | 8 | Citrate | 3 |
| | | | | | buffer solution | |
| OKI-179 | 100 | 40 | 10 | 4 | Citrate | 3 |
| | | | | | buffer solution | |

### Tolerability

bini/enco/OKI-179 80 mpk group-2 animals on dosing holiday (>10% BWL) Day 11, 12 and 1 animal found dead Day 12
bini/enco/OKI-179 40 mpk group-2 animals on dosing holiday (>10% BWL) Day 11, 12

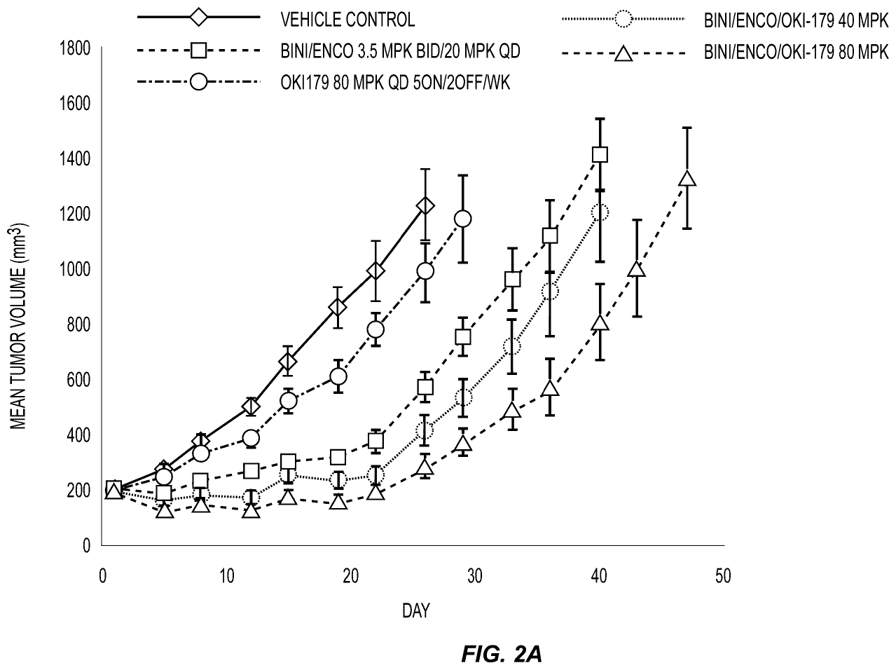

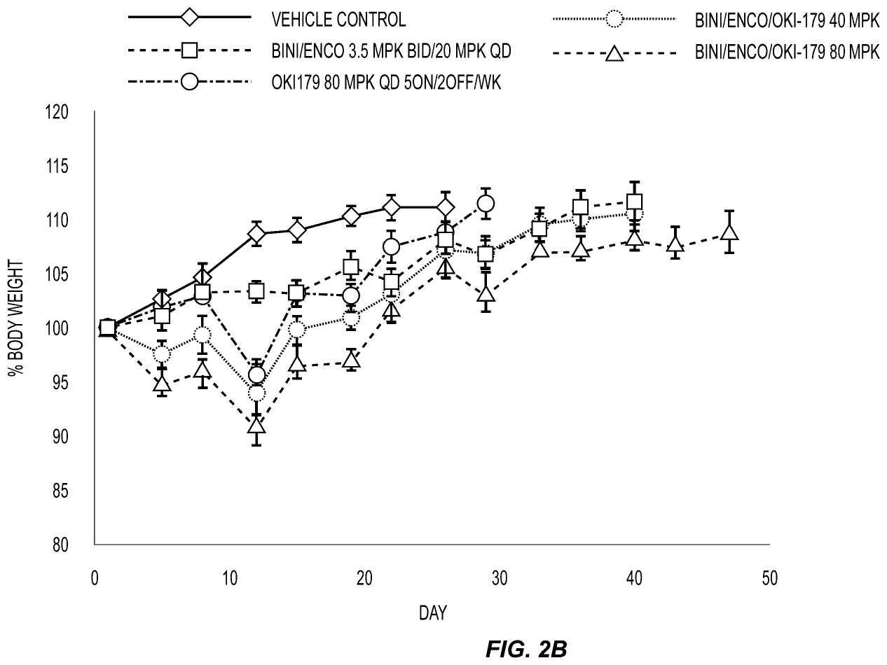

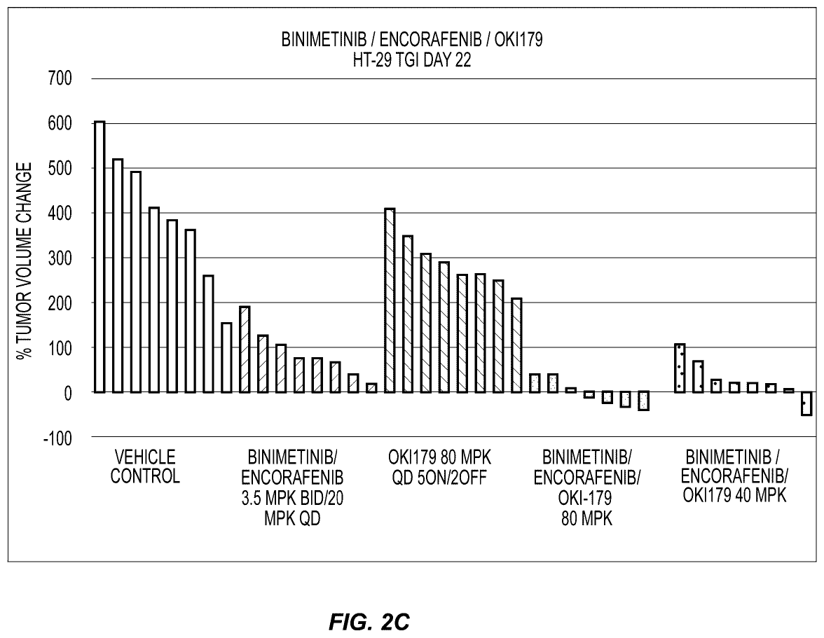

The results of this study are summarized in Table 1 and FIGS. 2A, 2B and 2C.

### Protocol 2

### CRC563 (BRAF V600E) Protocol

The OKI-179/bini/enco CRC563 (BRAF V600E) xenograft study was set up as follows:

### Study Design

vehicle control BID (days 1-14);
3.5 mpk bini BID (days 1-14)/20 mpk enco QD (days 1-14)
80 mpk OKI-179 QD1 (5 consecutive days on/2 consecutive days off for 14 days)
3.5 mpk bini BID (days 1-9)/20 mpk enco QD (days 1-9)/80 mpk OKI 179 QD (5 consecutive days on/2 consecutive days off for 14 days)
3.5 mpk bini BID (days 1-14)/20 mpk enco QD (days 1-14)/40 mpk OKI 179 QD (5 consecutive days on/2 days consecutive off for 14 days)

### Dosing Solutions

bini (100% active): 1% CMC/0.5% Tween 80 was added to bini to make a concentration of 0.35 mg/mL. The suspension was sonicated for 30 minutes and stored at 4° C.
bini (100% active)/enco (100% active): 1% CMC/0.5% Tween 80 was added to bini to make a concentration of 0.7 mg/ml and the suspension was sonicated for 30 minutes. 1% CMC/0.5%Tween 80 was added to enco to make a concentration of 4 mg/mL and the suspension was sonicated for 30 minutes. The bini and enco suspensions were mixed 1:1 for a final dosing suspension of 0.35 mg/mL bini and 2 mg/mL enco and the combined suspension was stored at 4° C.
OKI-179 (100% active):
   Citrate buffer solution: Solution A (0.1M citric acid) and Solution B (0.1M citric acid, tri-sodium) were mixed: 100 parts Solution A: 20 parts Solution B to provide citrate buffer pH3.
   80 mg/kg—dried OKI-179 was added to citrate buffer pH3 and the mixture was stirred until homogenous formulation of 8 mg/ml was obtained. The dose solution was stored at 4° C. and used within 10 days.

Table 3 below summarizes the above dosing solutions.

TABLE 3

| | | Dose | Dose | Conc. | | |
|---|---|---|---|---|---|---|
| API | % API | mg/kg | ml/kg | mg/ml | Vehicle | pH |
| bini | 100 | 3.5 | 10 | 0.35 | 1% CMC/0.5% | |
| | | | | | Tween 80 | |
| enco | 100 | 20 | 10 | 2 | 1% CMC/0.5% | |
| | | | | | Tween 80 | |
| OKI-179 | 100 | 80 | 10 | 8 | Citrate | 3 |
| | | | | | buffer solution | |
| OKI-179 | 100 | 40 | 10 | 4 | Citrate | 3 |
| | | | | | buffer solution | |

### Tolerability

bini/enco/OKI-179 80 mpk group-all animals on dosing holiday for OKI-179 (>10% BWL) Days 4, 5 and all dosing stopped on Day 9 (BWL)
OKI-179 80 mpk group-all animals on dosing holiday (>10% BWL) Day 5, Day 9 all dosing stopped (BWL)

The results of this study are summarized in Table 1.

### Protocol 3

### MM415 (NRAS Q61L) Protocol

The OKI-179/bini MM415 (NRAS Q61L) xenograft study was set up as follows:

### Study Design

vehicle control BID (days 1-28)
3.5 mpk bini BID (days 1-28)
80 mpk OKI-179 QD (5 consecutive days on/2 consecutive days off for 28 days)
3.5 mpk bini BID (days 1-28)/80 mpk OKI-179 QD (5 consecutive days on/2 consecutive days off for 28 days)
3.5 mpk bini BID (days 1-28)/40 mpk OKI-179 QD (5 consecutive days on/2 consecutive days off for 28 days)

### Dosing Solutions

bini (100% active): 1% CMC/0.5% Tween 80 was added to bini to make a concentration of 0.35 mg/mL. The suspension was sonicated for 30 minutes and stored at 4° C.
OKI-179 (OnKure) (100% active):
   Citrate buffer solution: Solution A (0.1M citric acid) and Solution B (0.1M citric acid, tri-sodium) were mixed: 100 parts Solution A: 20 parts Solution B to provide citrate buffer pH3.
   80 mg/kg—Dried OKI-179 was added to citrate buffer pH3 and stirred until a homogenous formulation of 8 mg/mL was obtained. The dose solution was stored at 4° C. and used within 10 days.

Table 4 below summarizes the above dosing solutions.

TABLE 4

| | | Dose | Dose | Conc. | | |
|---|---|---|---|---|---|---|
| API | % API | mg/kg | ml/kg | mg/ml | Vehicle | pH |
| bini | 100 | 3.5 | 10 | 0.35 | 1% CMC/0.5% | |
| | | | | | Tween 80 | |
| OKI-179 | 100 | 80 | 10 | 8 | Citrate | 3 |
| | | | | | buffer solution | |
| OKI-179 | 100 | 40 | 10 | 4 | Citrate | 3 |
| | | | | | buffer solution | |

### Tolerability

OKI-179 80 mpk group—2 animals on dosing holiday (>10% BWL) Day 26

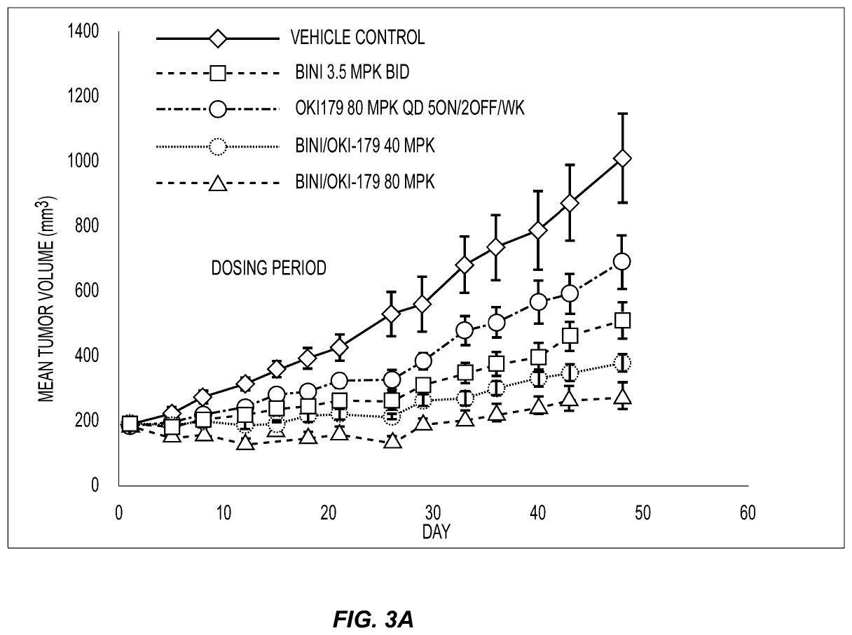

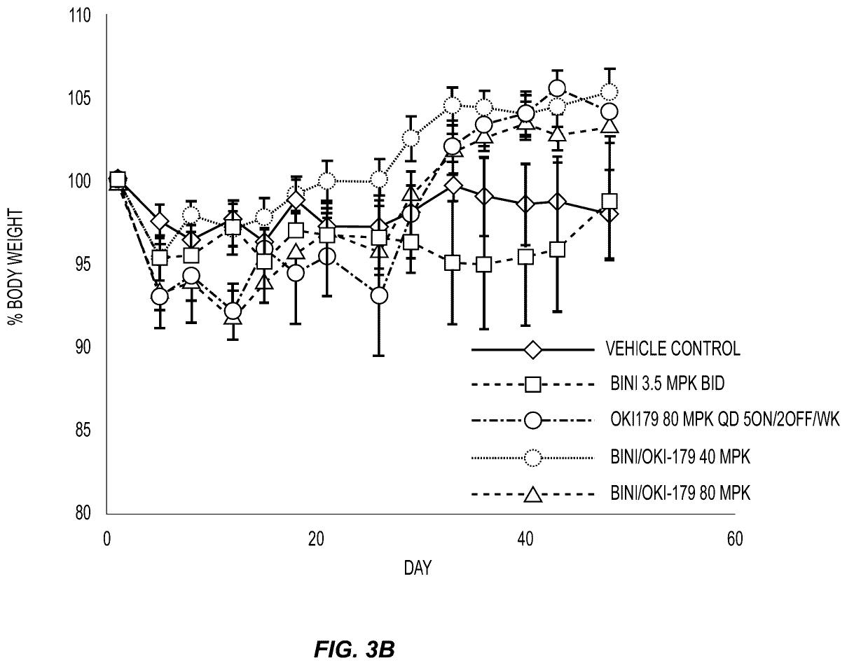

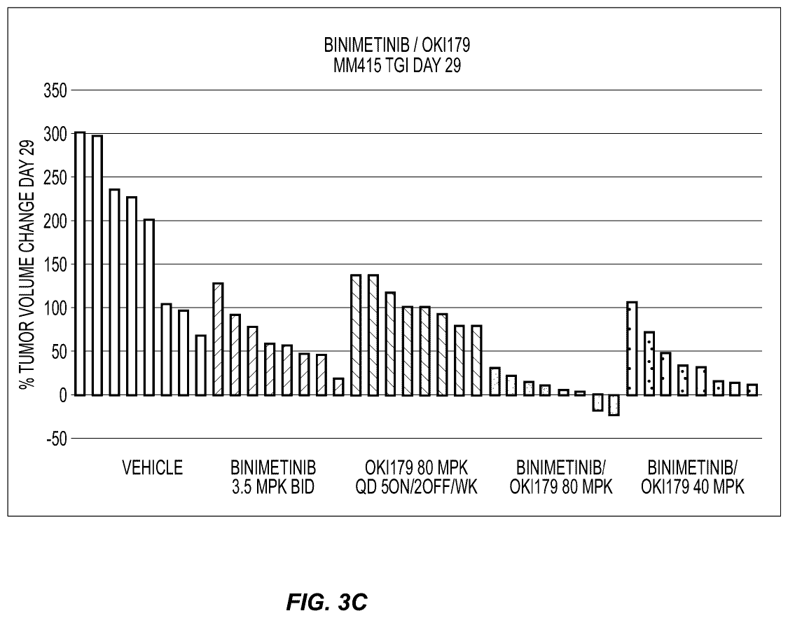

The results of this study are summarized in Table 1 and FIGS. 3A, 3B and 3C.

### Protocol 4

### MEL278 (NRAS Q61K) Protocol

The OKI-179/bini MEL278 (NRAS Q61K) xenograft study was set up as follows:

### Study Design

vehicle control BID (days 1-21)
3.5 mpk bini BID (days 1-21)
80 mpk OKI-179 QD (5 consecutive days on/2 consecutive days off for 21 days)
3.5 mpk bini BID (days 1-21)/80 mpk OKI 179 QD (5 consecutive days on/2 consecutive days off for 21 days)
3.5 mpk bini BID (days 1-21)/40 mpk OKI 179 QD (5 consecutive days on/2 consecutive days off for 21 days)

### Dosing Solutions

bini (100% active): 1% CMC/0.5% Tween 80 was added to bini to make a concentration of 0.35 mg/mL. The suspension was sonicated for 30 minutes and stored at 4° C.
OKI-179 (OnKure) (100% active):
   Citrate buffer solution: Solution A (0.1M citric acid) and Solution B (0.1M citric acid, tri-sodium) were mixed: 100 parts Solution A: 20 parts Solution B to provide citrate buffer pH3.
   80 mg/kg—Dried OKI-179 was added to citrate buffer pH3 and stirred until homogenous formulation of 8 mg/mL was obtained. The dose solution was stored at 4° C. and used within 10 days.

Table 5 below summarizes the above dosing solutions.

TABLE 5

| | | Dose | Dose | Conc. | | |
|---|---|---|---|---|---|---|
| API | % API | mg/kg | ml/kg | mg/ml | Vehicle | pH |
| bini | 100 | 3.5 | 10 | 0.35 | 1% CMC/0.5% | |
| | | | | | Tween 80 | |
| OKI-179 | 100 | 80 | 10 | 8 | Citrate | 3 |
| | | | | | buffer solution | |
| OKI-179 | 100 | 40 | 10 | 4 | Citrate | 3 |
| | | | | | buffer solution | |

### Tolerability

OKI-179 80 mpk group-1 animal found dead Day 12
bini/OKI179 80 mpk-all animals dosing holiday Day 8, 9; 1 animal found dead Day 7

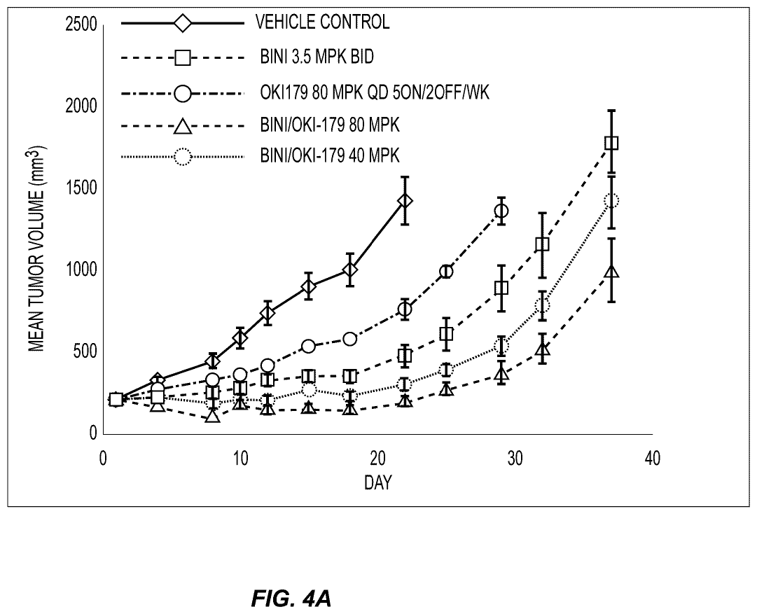

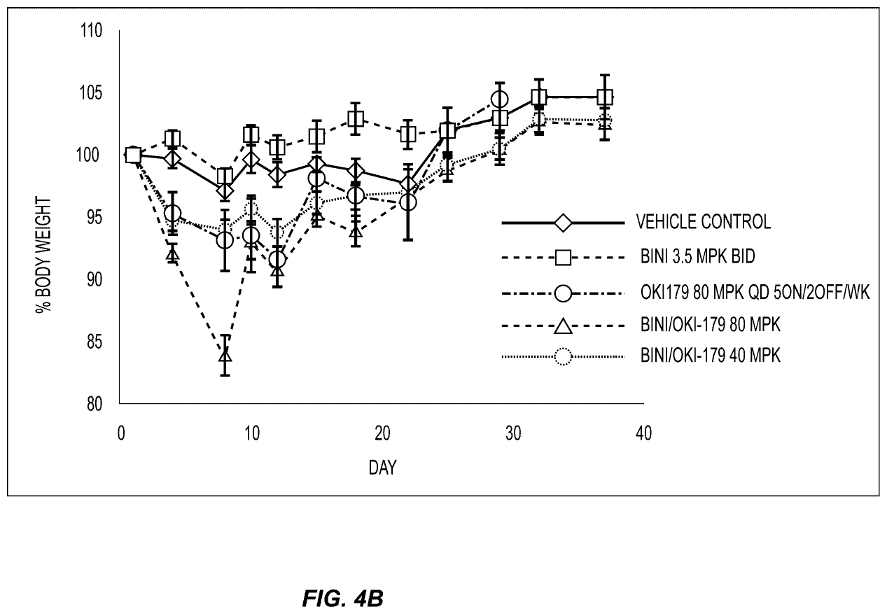

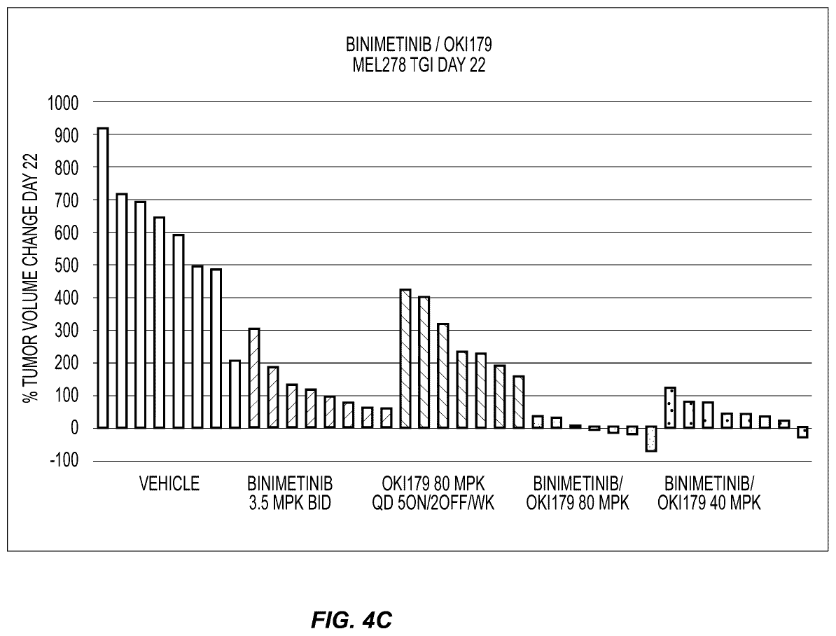

The results of this study are summarized in Table 1 and FIGS. 4A, 4B and 4C.

### Protocol 5

### SKMEL2 (NRAS Q61R) CDX Model Protocol

SKMEL2 human melanoma cells were grown as monolayers in tissue culture, harvested, resuspended in 50% DMEM/50% Matrigel, and 107 cells/mouse (100 ul of a 108 cells/ml suspension) were implanted subcutaneously in the right flank of female NOD-SCID mice. Tumor length and width were measured by caliper, and tumor volume calculated using the formula: volume (in mm³)=(length×width²)/2. When tumor volume reached an average size of approximately 170 mm³ (22 days after implantation), mice were randomized into treatment groups of 7 mice per group, with a tumor size range from 153 to 200 mm³. OKI-179 was formulated in 0.1 M citrate buffer pH 2.8-3.0 at 5 mg/mL and administered by oral gavage once a day (QD), 5 days on/2 days off/week, at a volume of 16 ml/kg (80 mg/kg dose). During the third week of treatment, OKI-179 was dosed at 60mg/kg. Binimetinib was formulated in 0.5% Tween-80/1% CMC at 0.35 mg/ml and administered by oral gavage twice a day (BID) at a volume of 10 ml/kg (3.5mg/kg dose). Tumor volume was measured twice a week. Animal weights were measured daily. If animal weight dropped to 85% of the weight at randomization (starting weight), treatment was stopped and then restarted after the weight recovered.

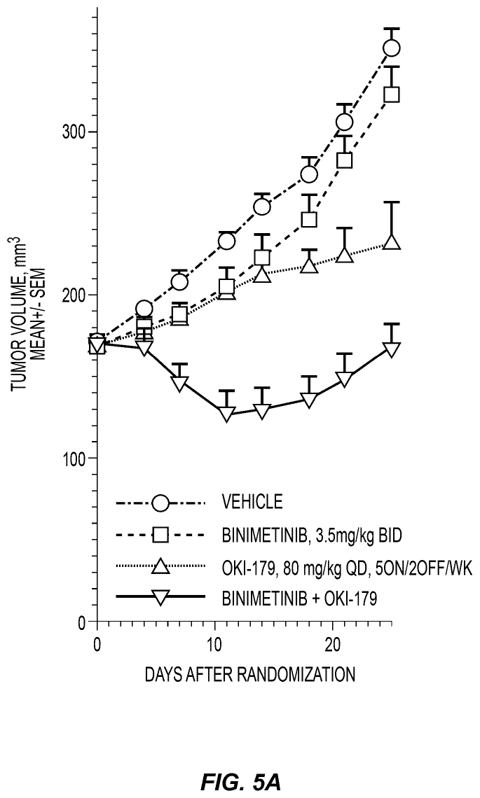

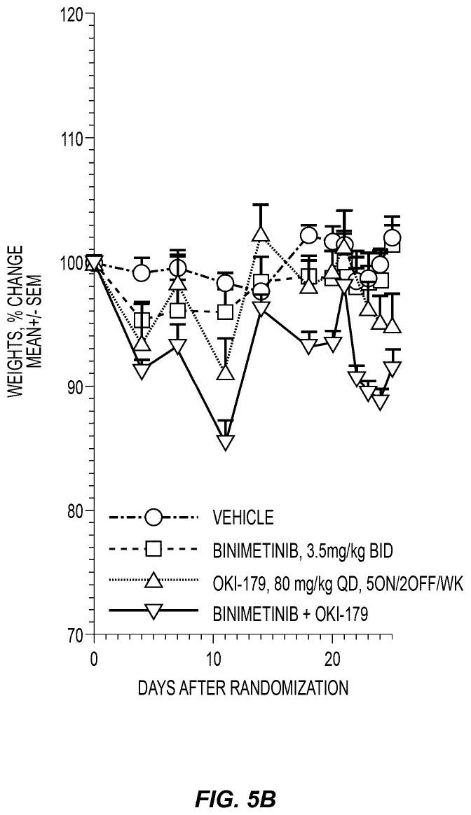

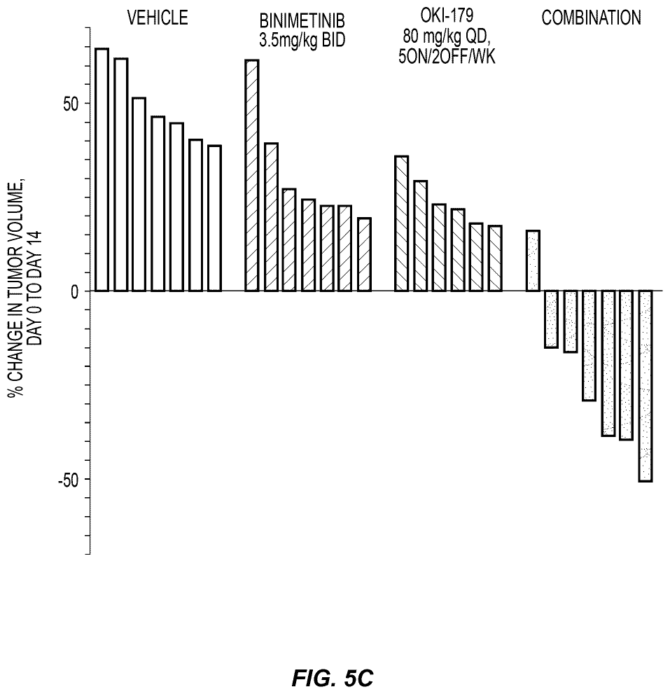

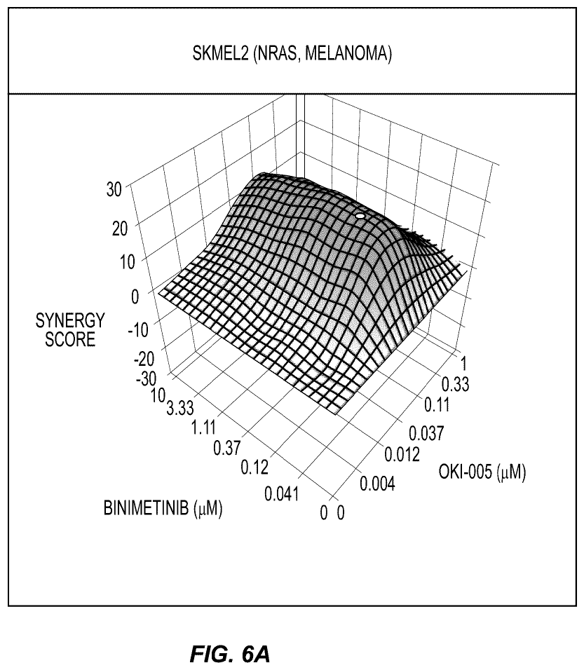

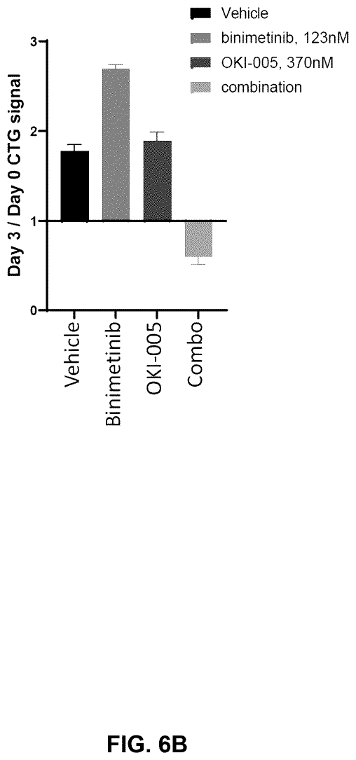

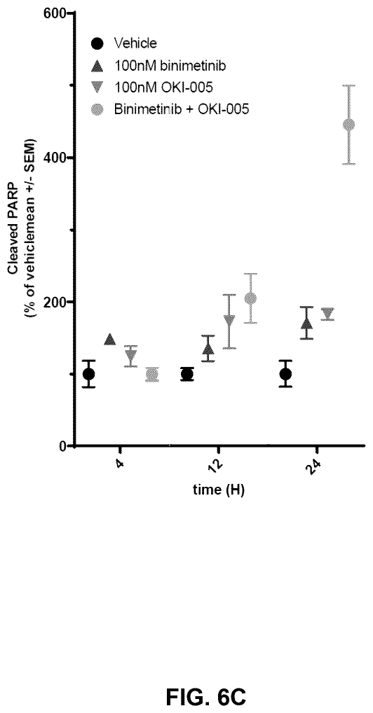

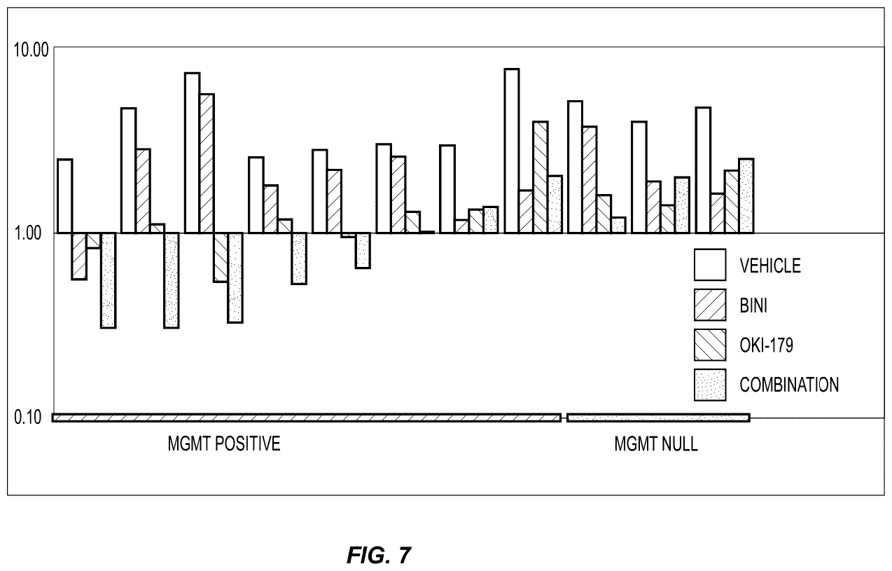

The results of this study are summarized in Table 1 and FIGS. 5A, 5B and 5C.

In the above Protocols 1-5, OKI-179 at a concentration of 80 mpk had a dosing holiday in every study. The combination of OKI-179 at 40 mpk and 80 mpk with binimetinib and with binimetinib+encorafenib was observed to exhibit increased TGI as compared to OKI-179 80 mpk or bini as a single agent in the NRAS melanoma models or bini/enco in the HT-29 study.

1. A method of treating cancer comprising administering to a subject in need thereof therapeutically effective amounts of OKI-179 and binimetinib or a pharmaceutically acceptable salt thereof according to a dosing schedule comprising at least one 7-day dosing cycle, wherein OKI-179 is administered once per day (QD) on days 1-4 of the 7-day dosing cycle and binimetinib or a pharmaceutically acceptable salt thereof is administered twice per day (BID) on days 1-7 of the 7-day dosing cycle.

2. A method of treating cancer comprising administering to a subject in need thereof therapeutically effective amounts of OKI-179 and binimetinib or a pharmaceutically acceptable salt thereof according to a dosing schedule comprising at least one 7-day dosing cycle, wherein OKI-179 is administered once per day (QD) on days 1-5 of the 7-day dosing cycle and binimetinib or a pharmaceutically acceptable salt thereof is administered twice per day (BID) on days 1-7 of the 7-day dosing cycle.

3. A method of treating cancer comprising administering to a subject in need thereof therapeutically effective amounts of OKI-179, binimetinib or a pharmaceutically acceptable salt thereof, and encorafenib or a pharmaceutically acceptable salt thereof according to a dosing schedule comprising at least one 7-day dosing cycle, wherein OKI-179 is administered once per day (QD) on days 1-4 of the 7-day dosing cycle, binimetinib or a pharmaceutically acceptable salt thereof is administered twice per day (BID) on days 1-7 of the 7-day dosing cycle and encorafenib or a pharmaceutically acceptable salt thereof is administered once per day (QD) on days 1-7 of the 7-day dosing cycle.

4. A method of treating cancer comprising administering to a subject in need thereof therapeutically effective amounts of OKI-179, binimetinib or a pharmaceutically acceptable salt thereof, and encorafenib or a pharmaceutically acceptable salt thereof according to a dosing schedule comprising at least one 7-day dosing cycle, wherein OKI-179 is administered once per day (QD) on days 1-5 of the 7-day dosing cycle, binimetinib or a pharmaceutically acceptable salt thereof is administered twice per day (BID) on days 1-7 of the 7-day dosing cycle and encorafenib or a pharmaceutically acceptable salt thereof is administered once per day (QD) on days 1-7 of the 7-day dosing cycle.

5. The method of claim 1, wherein the cancer is a NRAS-mutated cancer or a BRAF-mutated cancer.

6. The method of claim 1, wherein the subject is a human.

7. The method of claim 1, wherein OKI-179 and binimetinib are administered within 30 minutes of each other.

8. The method of claim 3, wherein OKI-179, binimetinib and encorafenib are administered within 30 minutes of each other.

9. The method of claim 1, wherein OKI-179 and binimetinib are administered orally.

10. The method of claim 3, wherein OKI-179, binimetinib and encorafenib are administered orally.

11. A pharmaceutical combination comprising OKI-179 and binimetinib or a pharmaceutically acceptable salt thereof, and optionally encorafenib or a pharmaceutically acceptable salt thereof.

12. (canceled)

13. The method of claim 2, wherein the cancer is a NRAS-mutated cancer or a BRAF-mutated cancer. 14 (New) The method of claim 3, wherein the cancer is a NRAS-mutated cancer or a BRAF-mutated cancer.

15. The method of claim 4, wherein the cancer is a NRAS-mutated cancer or a BRAF-mutated cancer. 16 (New) The method of claim 2, wherein OKI-179 and binimetinib are administered orally.

17. The method of claim 3, wherein OKI-179 and binimetinib are administered orally.

18. The method of claim 4, wherein OKI-179 and binimetinib are administered orally.

19. The method of claim 4, wherein OKI-179, binimetinib and encorafenib are administered orally. Page 6

20. The method of claim 2, wherein the subject is a human.

21. The method of claim 3, wherein the subject is a human.

22. The method of claim 4, wherein the subject is a human.
